SEQUENCE LISTING (18 sequences)

SEQ ID NO: 1 - DNA, 18 bp, synthetic construct
  misc_feature 1..18: note = primer PEDV-LM-F3
  source 1..18: mol_type = other DNA
tctgtgatgggccgacag

SEQ ID NO: 2 - DNA, 20 bp, synthetic construct
  misc_feature 1..20: note = primer PEDV-LM-B3
  source 1..20: mol_type = other DNA
ccagtgccagatgaagcatt

SEQ ID NO: 3 - DNA, 42 bp, synthetic construct
  misc_feature 1..42: note = primer PEDV-LM-FIP
  source 1..42: mol_type = other DNA
cctgtacgccagtagcaaccttgagcaccaactggtgtaacg

SEQ ID NO: 4 - DNA, 41 bp, synthetic construct
  misc_feature 1..41: note = primer PEDV-LM-BIP
  source 1..41: mol_type = other DNA
atttcgtcacagtcgccaaggcgactgaacgaccaacacgt

SEQ ID NO: 5 - DNA, 21 bp, synthetic construct
  misc_feature 1..21: note = primer PEDV-LS-F3
  source 1..21: mol_type = other DNA
aaatttaatgttcaggcacct

SEQ ID NO: 6 - DNA, 22 bp, synthetic construct
  misc_feature 1..22: note = primer PEDV-LS-B3
  source 1..22: mol_type = other DNA
gtggccttatgtaaataaagct

SEQ ID NO: 7 - DNA, 45 bp, synthetic construct
  misc_feature 1..45: note = primer PEDV-LS-FIP
  source 1..45: mol_type = other DNA
actagcagtttcaatgcctgtggttacctacctagtatgaactct

SEQ ID NO: 8 - DNA, 44 bp, synthetic construct
  misc_feature 1..44: note = primer PEDV-LS-BIP
  source 1..44: mol_type = other DNA
ggcgttcatggtatttttctcagcctaggatcaaacggctcttg

SEQ ID NO: 9 - DNA, 23 bp, synthetic construct
  misc_feature 1..23: note = primer PEDV-G1-F
  source 1..23: mol_type = other DNA
tgttttgggtggttatctaccta

SEQ ID NO: 10 - DNA, 19 bp, synthetic construct
  misc_feature 1..19: note = primer PEDV-G1-R
  source 1..19: mol_type = other DNA
agctggtaaccactaggat

SEQ ID NO: 11 - DNA, 24 bp, synthetic construct
  misc_feature 1..24: note = primer PEDV-G2-F
  source 1..24: mol_type = other DNA
ccagtactttcaacacttagccta

SEQ ID NO: 12 - DNA, 18 bp, synthetic construct
  misc_feature 1..18: note = primer PEDV-G2-R
  source 1..18: mol_type = other DNA
gccactagcagttggatg

SEQ ID NO: 13 - DNA, 20 bp, synthetic construct
  misc_feature 1..20: note = primer PEDV-MP-F
  source 1..20: mol_type = other DNA
ctgttgcgaactgttgagct

SEQ ID NO: 14 - DNA, 20 bp, synthetic construct
  misc_feature 1..20: note = primer PEDV-MP-R
  source 1..20: mol_type = other DNA
gccacgatcctgaaaactga

SEQ ID NO: 15 - DNA, 20 bp, synthetic construct
  misc_feature 1..20: note = primer PEDV-SP-F
  source 1..20: mol_type = other DNA
ctggttgttcttaccagtac

SEQ ID NO: 16 - DNA, 20 bp, synthetic construct
  misc_feature 1..20: note = primer PEDV-SP-R
  source 1..20: mol_type = other DNA
acatcattaacagtagggcc

SEQ ID NO: 17 - DNA, 20 bp, synthetic construct
  misc_feature 1..20: note = primer PEDV-F
  source 1..20: mol_type = other DNA
gtattggtggtgagcggaat

SEQ ID NO: 18 - DNA, 20 bp, synthetic construct
  misc_feature 1..20: note = primer PEDV-R
  source 1..20: mol_type = other DNA
cctgttccgccattctatca

(19) United States
(12) Patent Application Publication
(10) Pub. No.: US 2026/0103767 A1
(43) Pub. Date: Apr. 16, 2026

(54) Primer set and kit for simultaneous identification of classical strain and variant strain of porcine epidemic diarrhea virus (PEDV)

(71) Applicant: Jiangxi Agricultural University, Nanchang City (CN)

(72) Inventors: Deping SONG, Nanchang City (CN); Zhong LIU, Nanchang City (CN); Mengtao FANG, Nanchang City (CN); Jieqiong LI, Nanchang City (CN); Qi WU, Nanchang City (CN); Yu YE, Nanchang City (CN); Dongyan HUANG, Nanchang City (CN); Gen WAN, Nanchang City (CN)

(21) Appl. No.: 19/352,987

(22) Filed: Oct. 8, 2025

(30) Foreign Application Priority Data
Oct. 12, 2024 (CN) 202411420119.4

(51) Int. Cl. C12Q 1/70; C12Q 1/6848

(52) U.S. Cl. CPC C12Q 1/701; C12Q 1/6848; C12Q 2600/156

(57) ABSTRACT

A primer set and a kit is used for simultaneous identification of a classical strain and a variant strain of a porcine epidemic diarrhea virus (PEDV). The primer set includes primer set I and primer set II; where primer set I comprises the nucleotide sequences set forth in SEQ ID NO: 1 to SEQ ID NO: 4; and primer set II comprises the nucleotide sequences set forth in SEQ ID NO: 5 to SEQ ID NO: 8. This approach not only helps avoid false negatives or false positives in the detection of PEDV variant strain infections, but also enables the identification between classical strain and variant strain in PEDV. This is of significant importance for understanding the prevalence of PEDV and for the prevention and control of PEDV infections.

### CROSS-REFERENCE TO RELATED APPLICATION

This patent application claims the benefit and priority of Chinese Patent Application No. 202411420119.4 filed with the China National Intellectual Property Administration on Oct. 12, 2024, the disclosure of which is incorporated by reference herein in its entirety as part of the present application.

### REFERENCE TO SEQUENCE LISTING

A computer readable XML file entitled “GWP20250902246-sequence listing”, which was created on Nov. 10, 2025, with a file size of about 23,792 bytes, contains the sequence listing for this application, has been filed with this application, and is hereby incorporated by reference in its entirety.

### TECHNICAL FIELD

The present disclosure belongs to the technical field of biological detection, and specifically relates to a primer set and a kit for simultaneous identification of a classical strain and a variant strain of a porcine epidemic diarrhea virus (PEDV), and more specifically to a reverse transcription loop-mediated isothermal amplification (RT-LAMP)-based primer set, a kit, and a detection method for rapid and simultaneous identification of a classical strain and a variant strain of a PEDV.

### BACKGROUND

Porcine epidemic diarrhea virus (PEDV) is the pathogen responsible for a highly contagious intestinal disease in pigs. Porcine epidemic diarrhea (PED) is characterized by diarrhea in pigs of all age groups, with vomiting, diarrhea, reduced appetite, and death in piglets within one week of age being the primary symptoms. Pigs are the only natural hosts of PEDV, and this virus can infect pigs of all age stages, including newborn piglets, fattening pigs, sows, and boars. The severity of the damage caused by PEDV is closely related to the age of the pigs, with lesser harm to fattening pigs and breeding pigs, though it can cause diarrhea and agalactosis in sows and affect their reproductive cycles. However, the impact on suckling piglets, particularly newborn piglets within 7 days of age, is relatively more severe. The main sources of PEDV infection are virus-carrying or infected pigs, followed by contaminated feed, packaging materials, vehicles, shoes, clothing, and other items that can spread the virus. Fecal-oral transmission is the primary route of PEDV transmission. In recent years, this virus has also been detected in the milk of sows, suggesting that sow milk may also be a transmission route for PEDV. Additionally, some reports indicate that this virus may be transmitted through the respiratory tract and can be shed via respiratory secretions. This means that within the same or adjacent pig pens, PEDV may primarily spread through direct contact with healthy pigs, as well as through indirect contact, feeding on virus-contaminated feed and water, and other means of horizontal transmission.

Extensive genomic sequencing and analysis have revealed the presence of both the variant strain and classical strain of PEDV in clinical infections. Currently, the prevalent PEDV strain is mainly variant strain (genotype G2), which differs significantly from the classical strain (genotype G1, represented by the CV777 strain) in terms of genotype and genetic evolution. Furthermore, due to substantial differences in antigenicity, immunogenicity, and protective efficacy between the variant strain and classical strain, vaccines based on the PEDV classical strain provide poor protection against PEDV variant strain, making the prevention and control of PED clinically challenging. There are also significant differences in pathogenicity between the variant strain and classical strain of PEDV. In field conditions, infections involving both the variant strain and classical strain are difficult to distinguish based on symptoms and lesions alone, requiring laboratory diagnosis to differentiate the genotypes of the strains. Currently, laboratory diagnostic methods for PEDV include reverse transcription PCR (RT-PCR), real-time fluorescent quantitative RT-PCR, enzyme-linked immunosorbent assay (ELISA), neutralization tests, virus isolation and identification, immunoelectron microscopy, immunofluorescence, etc. However, existing diagnostic methods cannot directly differentiate whether a PEDV strain is a classical strain or a variant strain. Traditional methods for differentiating the classical strain and variant strain of PEDV involve gene sequencing and comparative analysis, which are time-consuming and labor-intensive. Therefore, there is an urgent need for a rapid detection method that can both detect PEDV and differentiate between classical strain and variant strain for non-diagnostic purposes.

### SUMMARY

In view of the deficiencies of the prior art, a first object of the present disclosure is to provide a primer set for simultaneous identification of a classical strain and a variant strain of a PEDV.

A second object of the present disclosure is to provide a reverse transcription loop-mediated isothermal amplification (RT-LAMP)-based kit for rapidly and simultaneously detecting a PEDV and differentiating between a classical strain and a variant strain of the PEDV.

A third object of the present disclosure is to provide an RT-LAMP-based detection method for rapidly and simultaneously detecting a PEDV and differentiating between a classical strain and a variant strain of the PEDV for a non-diagnostic purpose.

A fourth object of the present disclosure is to provide the use of the RT-LAMP-based kit in detection of a PEDV and differentiation between a classical strain and a variant strain of the PEDV for a non-diagnostic purpose.

The present disclosure adopts the following technical solutions:

The present disclosure provides an RT-LAMP-based primer set for simultaneous identification of a classical strain and a variant strain of a PEDV, including: primer set I and primer set II; where
primer set I includes outer primer pair I consisting of PEDV-LM-F3 and PEDV-LM-B3, and inner primer pair I consisting of PEDV-LM-FIP and PEDV-LM-BIP; and primer set I comprises the nucleotide sequences set forth in SEQ ID NO: 1 to SEQ ID NO: 4; and
primer set II includes outer primer pair II consisting of PEDV-LS-F3 and PEDV-LS-B3, and inner primer pair II consisting of PEDV-LS-FIP and PEDV-LS-BIP; and primer set II comprises the nucleotide sequences set forth in SEQ ID NO: 5 to SEQ ID NO: 8. Specifically, each sequence has the following nucleotide sequence (5′-3′):

PEDV-LM-F3:
(SEQ ID NO: 1)
TCTGTGATGGGCCGACAG;
PEDV-LM-B3:
(SEQ ID NO: 2)
CCAGTGCCAGATGAAGCATT;
PEDV-LM-FIP:
(SEQ ID NO: 3)
CCTGTACGCCAGTAGCAACCTTGAGCACCAACT GGTGTAACG;
PEDV-LM-BIP:
(SEQ ID NO: 4);
ATTTCGTCACAGTCGCCAAGGCGACTGAACGA CCAACACGT
PEDV-LS-F3:
(SEQ ID NO: 5)
AAATTTAATGTTCAGGCACCT;
PEDV-LS-B3:
(SEQ ID NO: 6)
GTGGCCTTATGTAAATAAAGCT;
PEDV-LS-FIP:
(SEQ ID NO: 7)
ACTAGCAGTTTCAATGCCTGTGGTTACCTACCTAG-
TATGAACTCT;
PEDV-LS-BIP:
(SEQ ID NO: 8)
GGCGTTCATGGTATTTTTCTCAGCCTAGGATCAAAC-
GGCTCTTG.

Further, the universal RT-LAMP-based detection primer set for detecting the classical strain and the variant strain of the PEDV includes two pairs of specific primers in primer set I, namely outer primer pair I consisting of PEDV-LM-F3 and PEDV-LM-B3 and inner primer pair I consisting of PEDV-LM-FIP and PEDV-LM-BIP. The RT-LAMP-based detection primer set for distinguishing the classical strain from the variant strain of the PEDV includes two pairs of specific primers in primer set II, namely outer primer pair II of PEDV-LS-F3 and PEDV-LS-B3, and inner primer pair TT of PEDV-LS-FIP and PEDV-LS-BIP.

The present disclosure further provides a kit for simultaneous identification of a classical strain and a variant strain of a PEDV, including: the RT-LAMP-based primer set, a reverse transcriptase, an RT-LAMP reaction solution, a positive control, and a negative control.

Further, outer primer pair I and inner primer pair I are at a molar ratio of 1:8.

Further, outer primer pair II and inner primer pair II are at a molar ratio of 1:8.

The present disclosure further provides a method for simultaneous identification of a classical strain and a variant strain of a PEDV for a non-diagnostic purpose, including the following steps:
(1) conducting RNA extraction and reverse transcription: extracting a viral nucleic acid from a sample, and subjecting an RNA to reverse transcription to obtain a sample cDNA using a reverse transcription kit;
(2) conducting an RT-LAMP-based amplification reaction: preparing an RT-LAMP-based amplification reaction system by mixing the kit with the sample cDNA, and conducting the RT-LAMP-based amplification reaction; and
(3) conducting result determination: adding a saturated fluorescent dye to a system obtained after the RT-LAMP-based amplification reaction to conduct visual RT-LAMP-based detection, and verifying an obtained visual RT-LAMP-based detection result using agarose gel electrophoresis.

Further, the RT-LAMP-based amplification reaction system includes a universal LAMP reaction system and an identification LAMP reaction system;
the universal LAMP reaction system, in a 25 μL volume, includes:
2.5 μL of 10× isothermal amplification buffer (containing 20 mM MgSO₄),
1.0 μL of 100 mM MgSO₄,
2.5 μL of 10 mM dNTP,
1 μL of 8,000 U/mL Bst 2.0 DNA polymerase,
2.0 μL of 5 μM outer primer pair I,
2.0 μL of 40 μM inner primer pair I,
2.5 μL of sample cDNA, and
11.5 μL of sterilized distilled water; and a program includes: a reaction at 65° C. for 40 min, a reaction at 80° C. for 20 min, and inactivation in sequence; and
the identification LAMP reaction system, in a 25 μL volume, includes:
2.5 μL of 10× isothermal amplification buffer (containing 20 mM MgSO₄),
1.5 μL of 100 mM MgSO₄,
3.5 μL of 10 mM dNTP,
1.0 μL of 8,000 U/mL Bst 2.0 DNA polymerase,
2.0 μL of 5 μM outer primer pair II,
2.0 μL of 40 μM inner primer pair II, and
2.5 μL of sample cDNA, and
10.0 μL of sterilized distilled water; and a program includes: a reaction at 65° C. for 30 min, a reaction at 80° C. for 20 min, and inactivation in sequence.

The present disclosure further provides use of the kit in identification of a classical strain and a variant strain of a PEDV for a non-diagnostic purpose.

Compared with the prior art, the present disclosure has the following beneficial effects:
1. By using a relatively conserved M gene of PEDV as a target fragment and constructing a standard plasmid, a universal RT-LAMP-based rapid detection method for PEDV is established. By using an S gene of PEDV as a target fragment and constructing a standard plasmid, an identification RT-LAMP-based rapid detection method for PEDV is established. This approach not only helps avoid false negatives or false positives in the detection of PEDV variant strains, but also enables the identification between the classical strain and variant strain of PEDV. This is of significant importance for understanding the prevalence of PEDV and for the prevention and control of PEDV infections.
2. In the present disclosure, the classical strain and variant strain of PEDV are detected using the RT-LAMP-based method, which employs a specialized primer set, a kit containing the primer set; and a detection method adopts the kit. The kit is convenient to use and allows for the detection of the pathogen in a short time; the kit does not require large, complex, or expensive instruments; result identification is convenient and visual. After the reaction is completed, there is no need to open the tube to add additional reagents, thereby avoiding false positives caused by subsequent aerosol contamination due to uncapping of the tube. The kit eliminates the need for tedious post-reaction procedures such as nucleic acid electrophoresis. The entire process, including amplification and result determination, can be completed rapidly, accurately, and efficiently in just 40 min. The detection exhibits desirable specificity and high sensitivity. According to experimental comparative tests, the detection limit of the kit for detecting PEDV strains (including the classical strain and variant strain) can reach 10² copies per reaction, which is 100 to 1,000 times more sensitive than conventional PCR methods.
3. In the present disclosure, the detection provides reliable results at a relatively low cost, and features simplicity, rapidness, specificity, and sensitivity. It can meet the demands for rapid detection and provides a basis for the rapid detection of classical strain and variant strain of PEDV.

### BRIEF DESCRIPTION OF THE DRAWINGS

FIG. 1A-FIG. 1E show the universal RT-LAMP condition optimization results of PEDV in the examples of the present disclosure; where FIG. 1A shows the electrophoretogram for MgSO₄ concentration optimization, FIG. 1B shows the electrophoretogram for dNTP concentration optimization, FIG. 1C shows the electrophoretogram for the optimization of the concentration ratio between the inner and outer primer I, FIG. 1D shows the electrophoretogram for reaction temperature optimization, FIG. 1E shows the electrophoretogram for reaction time optimization;

FIG. 2A-FIG. 2E show the identification RT-LAMP condition optimization results of PEDV in the examples of the present disclosure; where FIG. 2A shows the electrophoretogram for MgSO₄ concentration optimization, FIG. 2B shows the electrophoretogram for dNTP concentration optimization, FIG. 2C shows the electrophoretogram for the optimization of the concentration ratio between the inner and outer primer II, FIG. 2D shows the electrophoretogram for reaction temperature optimization, FIG. 2E shows the electrophoretogram for reaction time optimization;

FIG. 3A-FIG. 3D show the RT-LAMP specificity detection results of PEDV in the examples of the present disclosure; where FIG. 3A and FIG. 3B show the gel electrophoresis results and visual detection results of the universal RT-LAMP, respectively, FIG. 3C and FIG. 3D show the gel electrophoresis results and visual detection results of the identification RT-LAMP, respectively, + indicates positive, − indicates negative;

FIG. 4A-FIG. 4F show the RT-LAMP and PCR sensitivity detection results in the examples of the present disclosure; where FIG. 4A and FIG. 4B show the gel electrophoresis detection results of the universal RT-LAMP and identification RT-LAMP, respectively, FIG. 4C and FIG. 4D show the visual detection results of the universal RT-LAMP and identification RT-LAMP, respectively, FIG. 4E and FIG. 4F show the PCR sensitivity detection results for the PEDV G1 and PEDV G2 genotypes, respectively, + indicates positive, − indicates negative; and

FIG. 5 shows the RT-LAMP repeatability detection results in the examples of the present disclosure.

### DETAILED DESCRIPTION OF THE EMBODIMENTS

In the present disclosure, the various technical features described above and those specifically described below (e.g., in the examples) can be combined with each other to form new or preferred technical solutions. However, the present disclosure is not limited solely to these examples, and likewise, these examples do not restrict the present disclosure in any way.

The experimental methods in the following examples, unless otherwise specified, are conventional techniques in molecular biology, microbiology, recombinant DNA technology, immunology, and the like. The materials and reagents involved in the following examples, unless otherwise specified, are common commercially available products and can be purchased from the market.

PEDV genotype G1 positive, PEDV genotype G2 positive, and 33 unknown samples, as well as viral samples including Porcine Reproductive and Respiratory Syndrome Virus (PRRSV), Porcine Deltacoronavirus (PDCoV), Porcine Rotavirus (PoRV), and Pseudorabies Virus (PRV), were preserved by the Preventive Veterinary Laboratory of the College of Animal Science and Technology, Jiangxi Agricultural University. All samples were aliquoted and processed for DNA or RNA extraction, and the extracted nucleic acids were stored at −20° C. and −80° C. until use.

Bst 2.0 DNA polymerase (8,000 U/mL) were purchased from New England Biolabs (NEB, UK). dNTP mixture and the saturated fluorescent dye (4S Green Plus Nucleic Acid Stain) were purchased from Shanghai Sangon Biotech Engineering Services Co., Ltd. DL2000 DNA Marker, pMD19-T vector, RNA extraction reagents, and reverse transcription reagents were purchased from TaKaRa (Japan). Plasmid extraction kits and DNA extraction kits were purchased from TIANGEN Biotech (Beijing) Co., Ltd. Polymerase Chain Reaction Master Mix (PCR MasterMix) was purchased from Yeasen Biotech (Shanghai) Co., Ltd.

The present disclosure is further described below with reference to specific examples.

### Example 1: RT-LAMP-Based Primer Design

The M gene and S gene of different PEDV strains were compared and analyzed using Molecular Evolutionary Genetics Analysis version 11 (MEGA11) software. Targeting the most conserved region of the M gene and the hypervariable region of the S gene, universal RT-LAMP-based primers (primer set I) were designed using the online website Primer Explorer V5. The universal RT-LAMP-based primers included outer primer pair I, PEDV-LM-F3 and PEDV-LM-B3, and inner primer pair I, PEDV-LM-FIP and PEDV-LM-BIP. The nucleotide sequences in primer set I were set forth in SEQ ID NO: 1 to SEQ ID NO: 4 (5′-3′).

The identification RT-LAMP-based primers (primer set II) were designed, including outer primer pair II, PEDV-LS-F3 and PEDV-LS-B3, and inner primer pair II, PEDV-LS-FIP and PEDV-LS-BIP. The nucleotide sequences in primer set II were set forth in SEQ ID NO: 5 to SEQ ID NO: 8 (5′-3′):

PEDV-LM-F3:
(SEQ ID NO: 1)
TCTGTGATGGGCCGACAG;
PEDV-LM-B3:
(SEQ ID NO: 2)
CCAGTGCCAGATGAAGCATT;
PEDV-LM-FIP:
(SEQ ID NO: 3)
CCTGTACGCCAGTAGCAACCTTGAGCACCAACTGGTGTAACG;
PEDV-LM-BIP:
(SEQ ID NO: 4)
ATTTCGTCACAGTCGCCAAGGCGACTGAACGACCAACACGT;
PEDV-LS-F3:
(SEQ ID NO: 5)
AAATTTAATGTTCAGGCACCT;
PEDV-LS-B3:
(SEQ ID NO: 6)
GTGGCCTTATGTAAATAAAGCT;
PEDV-LS-FIP:
(SEQ ID NO: 7)
ACTAGCAGTTTCAATGCCTGTGGTTACCTACCTAG-
TATGAACTCT;
PEDV-LS-BIP:
(SEQ ID NO: 8)
GGCGTTCATGGTATTTTTCTCAGCCTAGGATCAAAC-
GGCTCTTG.

### Example 2: Extraction of Viral RNA and Construction of Plasmid Standards

Viral RNA from PEDV genotypes G1 and G2, as well as from PRRSV, PDCoV, and PoRV, was extracted according to the instructions for the RNA extraction reagents from TaKaRa. The extracted RNA was stored at −80° C. for future use. The aforementioned extracted viral RNA was reverse transcribed into cDNA using the reverse transcription kit from TaKaRa, and the cDNA was stored at −20° C. The DNA of PRV was extracted using the DNA extraction kit from TIANGEN according to the reagent instructions and stored at −20° C. for future use. Using the cDNA of PEDV genotypes G1 and G2 as templates, the M and S genes of PEDV were amplified using the following two sets of primers, PEDV-MP and PEDV-SP, respectively. The amplified products were then cloned into the pMD19-T vector for LAMP sensitivity tests. Using two sets of primers, PEDV-G1 and PEDV-G2, the genes were amplified and cloned into the pMD19-T vector for PCR sensitivity tests. All plasmids were extracted using the plasmid miniprep kit from TIANGEN, and the DNA concentration was measured using a NanoDrop 2000 spectrophotometer before being stored at −20° C. for future use. The nucleotide sequences of the respective primers are shown below:

PEDV-G1-F:
(SEQ ID NO: 9)
TGTTTTGGGTGGTTATCTACCTA;
PEDV-G1-R:
(SEQ ID NO: 10)
AGCTGGTAACCACTAGGAT;
PEDV-G2-F:
(SEQ ID NO: 11)
CCAGTACTTTCAACACTTAGCCTA;
PEDV-G2-R:
(SEQ ID NO: 12)
GCCACTAGCAGTTGGATG;
PEDV-MP-F:
(SEQ ID NO: 13)
CTGTTGCGAACTGTTGAGCT;
PEDV-MP-R:
(SEQ ID NO: 14)
GCCACGATCCTGAAAACTGA;
PEDV-SP-F:
(SEQ ID NO: 15)
CTGGTTGTTCTTACCAGTAC;
PEDV-SP-R:
(SEQ ID NO: 16)
ACATCATTAACAGTAGGGCC;
PEDV-F:
(SEQ ID NO: 17)
GTATTGGTGGTGAGCGGAAT;
PEDV-R:
(SEQ ID NO: 18)
CCTGTTCCGCCATTCTATCA.

### Example 3: Establishment and Optimization of the RT-LAMP-Based Reaction System

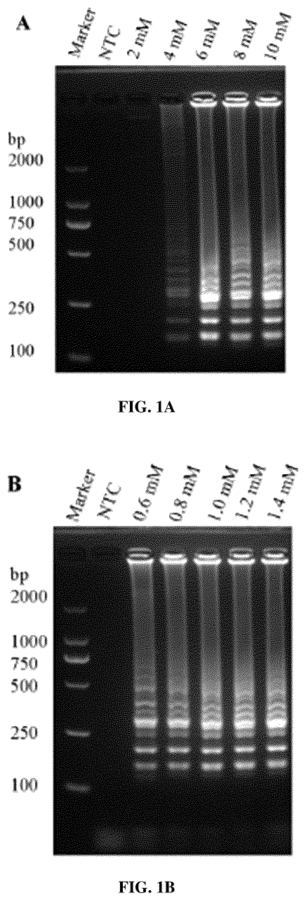

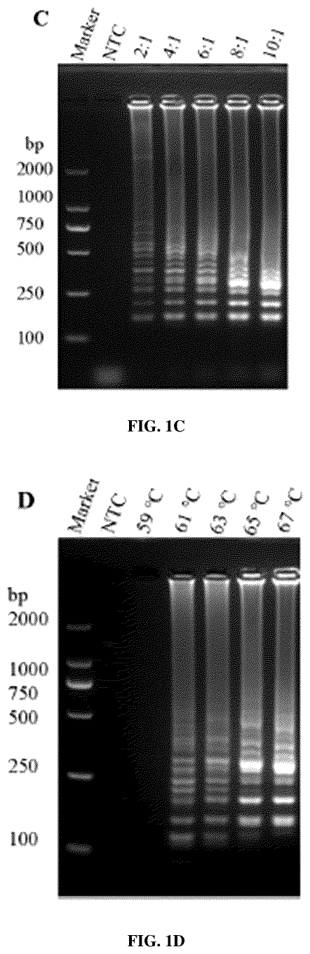

The universal RT-LAMP-based reaction was conducted using primer set I from Example 1 and a saturated fluorescent dye. The universal LAMP reaction system had a total volume of 25 μL, containing 2.5 μL of 10× isothermal amplification buffer (containing 20 mM MgSO₄), 1.0 μL of MgSO₄ (100 mM), 2.5 μL of dNTP (10 mM), 1.0 μL of Bst 2.0 DNA Polymerase (8,000 U/mL), 1.0 μL each of PEDV-LM-F3 (5 μM) and PEDV-LM-B3 (5 μM), 1.0 μL each of PEDV-LM-FIP (40 μM) and PEDV-LM-BIP (40 μM), 2.5 μL of template cDNA, and sterilized distilled water was added to bring the final volume to 25 μL. Optimization of the universal RT-LAMP-based reaction system was sequentially conducted from five aspects: Mg²⁺ concentration, dNTP concentration, ratio of inner to outer primer concentrations, reaction temperature, and reaction time, using the following procedures:
i) Mg²⁺ concentration optimization: the reaction system contained 100 mM MgSO₄ (0 μL, 0.5 μL, 1.0 μL, 1.5 μL, 2.0 μL), 2.5 μL of 10× isothermal amplification buffer (containing 20 mM MgSO₄), 2.5 μL of dNTP (10 mM), 1.0 μL of Bst 2.0 DNA Polymerase (8,000 U/mL), 1.0 μL each of PEDV-LM-F3 (5 μM) and PEDV-LM-B3 (5 μM), 1.0 μL each of PEDV-LM-FIP (40 μM) and PEDV-LM-BIP (40 μM), 2.5 μL of template cDNA, and sterilized distilled water was added to bring the final volume to 25 μL. The results indicated that the amplification efficiency was optimal when 1.0 μL of 100 mM MgSO₄ was added, resulting in a final Mg²⁺ concentration of 6 mM (FIG. 1A).
ii) dNTP concentration optimization: the reaction system contained 10 mM dNTP (2.0 μL, 2.5 μL, 3.0 μL, 3.5 μL, 4.0 μL), 2.5 μL of 10× isothermal amplification buffer (containing 20 mM MgSO₄), 1.0 μL of MgSO₄ (100 mM), 1.0 μL of Bst 2.0 DNA Polymerase (8,000 U/mL), 1.0 μL each of PEDV-LM-F3 (5 μM) and PEDV-LM-B3 (5 μM), 1.0 μL each of PEDV-LM-FIP (40 μM) and PEDV-LM-BIP (40 μM), 2.5 μL of template cDNA, and sterilized distilled water was added to bring the final volume to 25 μL. The results indicated that the amplification efficiency was optimal when 2.5 μL of 10 mM dNTP was added, resulting in a final dNTP concentration of 1 mM (FIG. 1B).
iii) Primer concentration optimization: the reaction system was set with a final concentration of outer primers I, PEDV-LM-F3 and PEDV-LM-B3, at 0.2 μM, and the final concentrations of inner primers I, PEDV-LM-FIP and PEDV-LM-BIP, were set at 0.4 μM, 0.8 μM, 1.2 μM, 1.6 μM, and 2.0 μM, respectively. Then, 2.5 μL of 10× isothermal amplification buffer (containing 20 mM MgSO₄), 1.0 μL of MgSO₄ (100 mM), 2.5 μL of dNTP (10 mM), 1.0 μL of Bst 2.0 DNA Polymerase (8,000 U/mL), 2.5 μL of template cDNA were sequentially added, and sterilized distilled water was added to bring the final volume to 25 μL. The results indicated that the amplification efficiency was optimal when the final concentration of the inner primers I was 1.6 μM, i.e., when the concentration ratio of inner primers I to outer primers I was 8:1 (FIG. 1C).
iv) Amplification temperature optimization: using the optimal amplification system described above, the amplification temperature was sequentially increased from 59° C., 61° C., 63° C., 65° C., to 67° C. The optimal reaction temperature was determined after multiple repetitions. The reaction time was sequentially increased from 40 min, 50 min, 60 min, 70 min, to 80 min. The optimal reaction time was determined after multiple repetitions. The results indicated that the optimal temperature for the universal LAMP reaction was 65° C. (FIG. 1D), and the optimal reaction time was 40 min (FIG. 1E).

The optimal reaction conditions for the universal LAMP reaction were finally determined as follows: 6 mM MgSO₄, 1 mM dNTP, a concentration ratio of inner primer I to outer primer I being 8:1, reacting at 65° C. for 40 min.

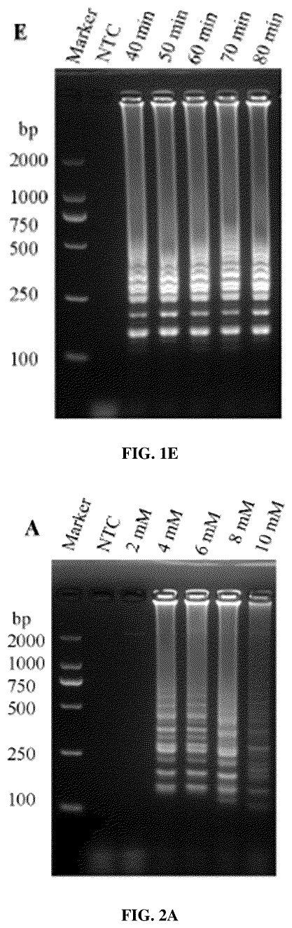

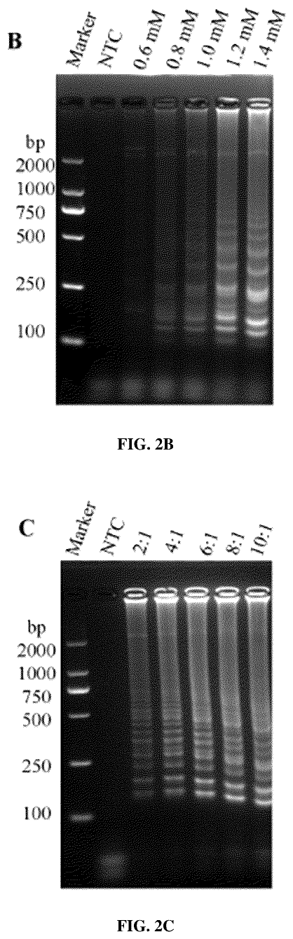

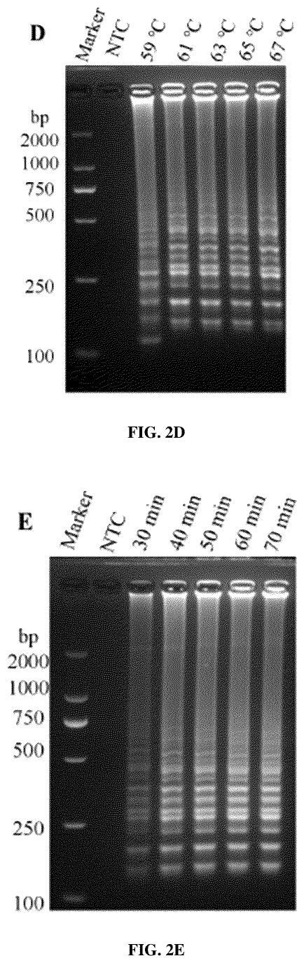

The identification RT-LAMP-based reaction was conducted using primer set II from Example 1 and a saturated fluorescent dye. The identification LAMP reaction system had a total volume of 25 μL, containing 2.5 μL of 10× isothermal amplification buffer (containing 20 mM MgSO₄), 1.5 μL of MgSO₄ (100 mM), 3.5 μL of dNTP (10 mM), 1.0 μL of Bst 2.0 DNA Polymerase (8,000 U/mL), 1.0 μL each of PEDV-LS-F3 (5 μM) and PEDV-LS-B3 (5 μM), 1.0 μL each of PEDV-LS-FIP (40 μM) and PEDV-LS-BIP (40 μM), 2.5 μL of template cDNA, and sterilized distilled water was added to bring the final volume to 25 μL. Optimization of the identification RT-LAMP-based reaction system was sequentially conducted from five aspects: Mg²⁺ concentration, dNTP concentration, ratio of inner primer to outer primer concentrations, reaction temperature, and reaction time, using the following procedures:
i) Mg²⁺ concentration optimization: the reaction system contained 100 mM MgSO₄ (0 μL, 0.5 μL, 1.0 μL, 1.5 μL, 2.0 μL), 2.5 μL of 10× isothermal amplification buffer (containing 20 mM MgSO₄), 3.5 μL of dNTP (10 mM), 1.0 μL of Bst 2.0 DNA Polymerase (8,000 U/mL), 1.0 μL each of PEDV-LS-F3 (5 μM) and PEDV-LS-B3 (5 μM), 1.0 μL each of PEDV-LS-FIP (40 μM) and PEDV-LS-BIP (40 μM), 2.5 μL of template cDNA, and sterilized distilled water was added to bring the final volume to 25 μL. The results indicated that the amplification efficiency was optimal when 1.5 μL of 100 mM MgSO₄ was added, resulting in a final Mg²⁺ concentration of 8 mM (FIG. 2A).
ii) dNTP concentration optimization: the reaction system contained 10 mM dNTP (2.0 μL, 2.5 μL, 3.0 μL, 3.5 μL, 4.0 μL), 2.5 μL of 10× isothermal amplification buffer (containing 20 mM MgSO₄), 1 μL of MgSO₄ (100 mM), 1.0 μL of Bst 2.0 DNA Polymerase (8,000 U/mL), 1.0 μL each of PEDV-LS-F3 (5 μM) and PEDV-LS-B3 (5 μM), 1.0 μL each of PEDV-LS-FIP (40 μM) and PEDV-LS-BIP (40 μM), 2.5 μL of template cDNA, and sterilized distilled water was added to bring the final volume to 25 μL. The results indicated that the amplification efficiency was optimal when 3.5 μL of 10 mM dNTP was added, resulting in a final dNTP concentration of 1.4 mM (FIG. 2B).
iii) Primer concentration optimization: the reaction system was set with a final concentration of outer primers II, PEDV-LS-F3 and PEDV-LS-B3, at 0.2 μM, and the final concentrations of inner primers II, PEDV-LS-FIP and PEDV-LS-BIP, were set at 0.4 μM, 0.8 μM, 1.2 μM, 1.6 μM, and 2.0 μM, respectively. Then, 2.5 μL of 10× isothermal amplification buffer (containing 20 mM MgSO₄), 1.5 μL of MgSO₄ (100 mM), 3.5 μL of dNTP (10 mM), 1.0 μL of Bst 2.0 DNA Polymerase (8,000 U/mL), 2.5 μL of template cDNA were sequentially added, and sterilized distilled water was added to bring the final volume to 25 μL. The results indicated that the amplification efficiency was optimal when the final concentration of the inner primers II was 1.6 μM, i.e., when the concentration ratio of inner primers II to outer primers II was 8:1 (FIG. 2C).
iv) Amplification temperature optimization: using the optimal amplification system described above, the amplification temperature was sequentially increased from 59° C., 61° C., 63° C., 65° C., to 67° C. The optimal reaction temperature was determined after multiple repetitions. The reaction time was sequentially increased from 30 min, 40 min, 50 min, 60 min, to 70 min. The optimal reaction time was determined after multiple repetitions. The results indicated that the optimal temperature for the identification LAMP reaction was 65° C. (FIG. 2D), and the optimal reaction time was 30 min (FIG. 2E).

The optimal reaction conditions for the identification LAMP reaction were finally determined as follows: 8 mM MgSO₄, 1.4 mM dNTP, an inner primer II to outer primer II concentration ratio being 8:1, reacting at 65° C. for 30 min.

### Example 4: RT-LAMP Specificity Test

The cDNA or DNA of PDCoV, PRRSV, PRV, and PoRV obtained in Example 2 was used as templates, while the cDNA of PEDV genotype G1 and PEDV genotype G2 served as positive controls, and distilled water served as a negative control. Amplification was conducted using the optimized LAMP reaction. After amplification, 5 μL of the reaction product was loaded onto a 2% agarose gel for electrophoresis detection, and additionally, 1 μL of fluorescent dye was added for visual observation.

The amplification reaction systems were as follows:

The universal LAMP reaction system, in a 25 μL volume, included:
2.5 μL of a 10× isothermal amplification buffer (containing 20 mM MgSO₄),
1.0 μL of 100 mM MgSO₄,
2.5 μL of 10 mM dNTP,
1.0 μL of 8,000 U/mL Bst 2.0 DNA Polymerase,
2.0 μL of 5 μM outer primer pair I,
2.0 μL of 40 μM inner primer pair I,
2.5 μL of sample cDNA, and
11.5 g L of sterilized distilled water; and a program included: a reaction at 65° C. for 40 min, a reaction at 80° C. for 20 min, and inactivation in sequence.

The identification LAMP reaction system, in a 25 μL volume, included:
2.5 μL of a 10× isothermal amplification buffer (containing 20 mM MgSO₄),
1.5 μL of 100 mM MgSO₄,
3.5 μL of 10 mM dNTP,
1.0 μL of 8,000 U/mL Bst 2.0 DNA Polymerase,
2.0 μL of 5 μM outer primer pair II,
2.0 μL of 40 μM inner primer pair II, and
2.5 μL of sample cDNA, and
10 μL of sterilized distilled water; and a program included: a reaction at 65° C. for 30 min, a reaction at 80° C. for 20 min, and inactivation in sequence.

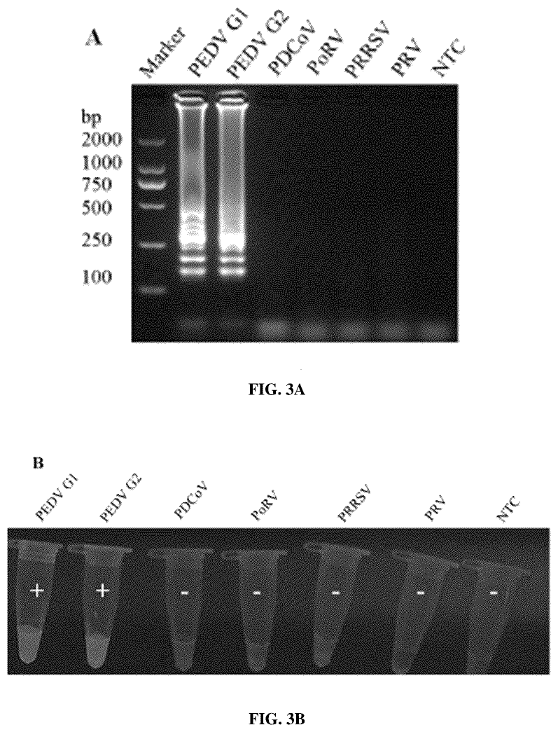

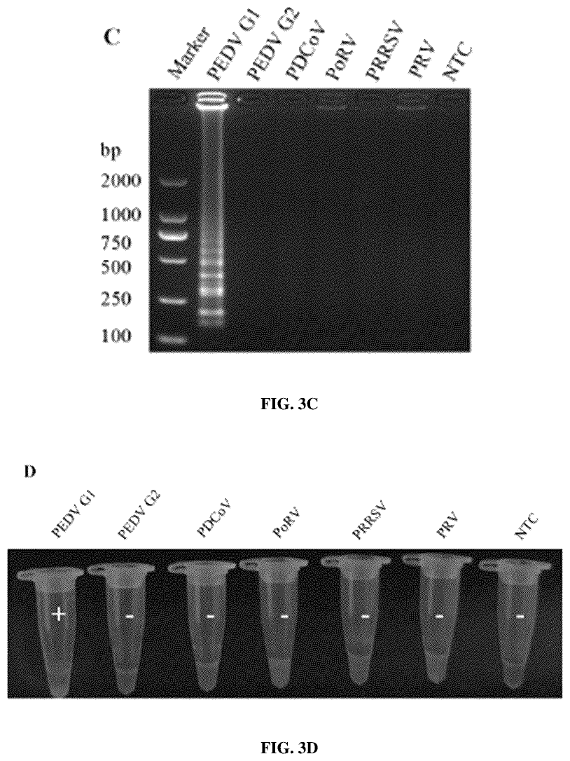

The results are shown in FIG. 3. Outer primer pair I, PEDV-LM-F3 and PEDV-LM-B3, and inner primer pair I, PEDV-LM-FIP and PEDV-LM-BIP, could only detect PEDV genotype G1 and PEDV genotype G2 viruses (FIG. 3A and FIG. 3B), but could not detect PDCoV, PoRV, PRRSV, or PRV. Outer primer pair II, PEDV-LS-F3 and PEDV-LS-B3, and inner primer pair II, PEDV-LS-FIP and PEDV-LS-BIP, could only detect PEDV genotype G1 virus, but could not detect PEDV genotype G2 virus, PDCoV, PoRV, PRRSV, or PRV (FIG. 3C and FIG. 3D).

### Example 5: RT-LAMP Sensitivity Test

The recombinant plasmids obtained in Example 2 were serially diluted 10-fold (from 1×10⁸ to 1×10⁰ copies) and used as templates for the LAMP and PCR sensitivity tests, respectively.

The LAMP reaction system was as that in Example 4. The PCR reaction system, in a 25 μL volume, included:
12.5 μL of a 2×PCR Master Mix,
1 μL of forward primer (SEQ ID NO: 13 or SEQ ID NO: 15),
1 μL of reverse primer (SEQ ID NO: 14 or SEQ ID NO: 16),
9.5 μL of sterilized distilled water, and
1 μL of recombinant plasmid.

The reaction program included: 94° C. for 5 min; 35 cycles of 94° C. for 10 s, 50° C. for 20 s, 72° C. for 15 s; and a final extension at 72° C. for 5 min.

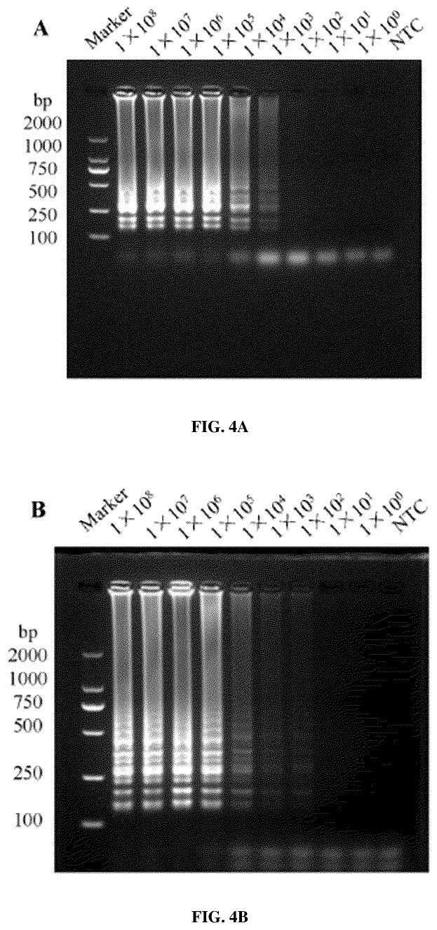

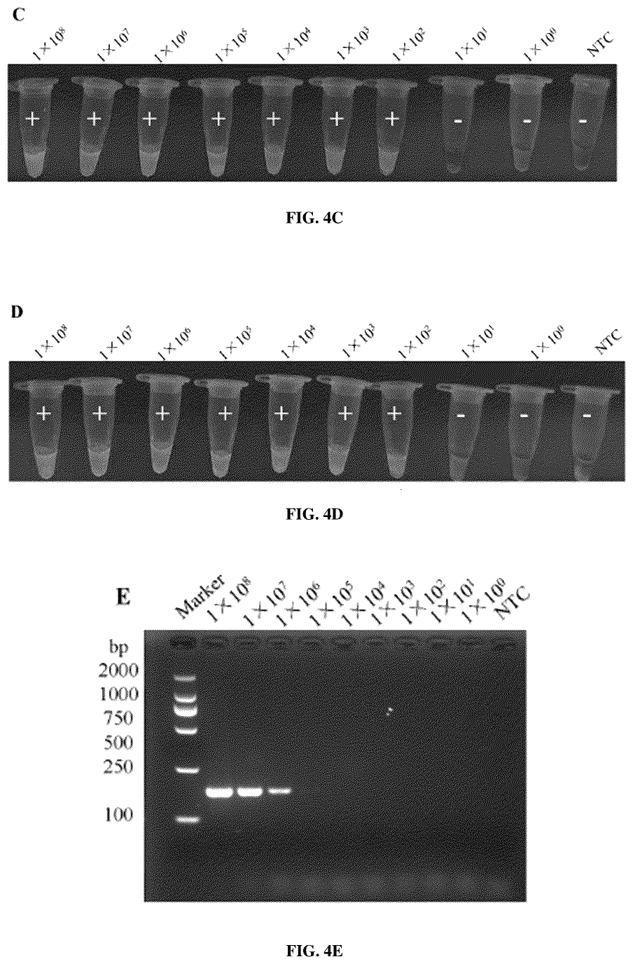

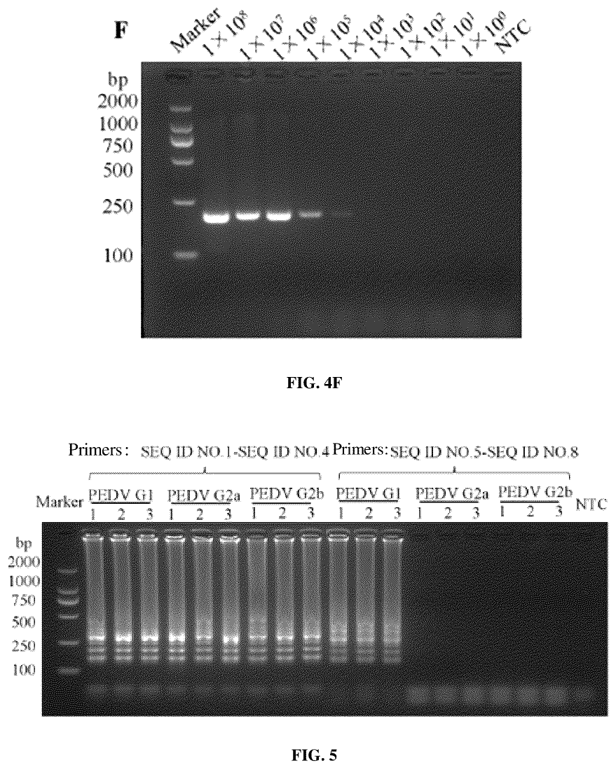

Based on the results from visual detection and gel electrophoresis analysis (FIG. 4), the lowest detection limit of the LAMP method for both PEDV genotype G1 and PEDV genotype G2 was 1×10² copies/reaction (FIG. 4A to FIG. 4D). In contrast, the lowest detection limits of the PCR method for PEDV genotype G1 and PEDV genotype G2 were 1×10⁵ copies/reaction and 1×10⁴ copies/reaction, respectively (FIG. 4E and FIG. 4F). The sensitivity of the LAMP method was 100 to 1,000 times higher than that of the PCR method.

### Example 6: RT-LAMP Repeatability Test

Using the method established in Example 3, the cDNA of PEDV genotype G1 and PEDV genotype G2 (including the 2 genetic subtypes, PEDV G2a and PEDV G2b) was used as templates. Three LAMP reactions were conducted: one using the universal primers, namely outer primer pair I PEDV-LM-F3 (SEQ ID NO: 1) and PEDV-LM-B3 (SEQ ID NO: 2), and inner primer pair I PEDV-LM-FIP (SEQ ID NO: 3) and PEDV-LM-BIP (SEQ ID NO: 4), and another using the identification primers, namely outer primer pair II PEDV-LS-F3 (SEQ ID NO: 5) and PEDV-LS-B3 (SEQ ID NO: 6), and inner primer pair II PEDV-LS-FIP (SEQ ID NO: 7) and PEDV-LS-BIP (SEQ ID NO: 8). As shown in FIG. 5, the PEDV genotype G1 could be detected by both primer sets. However, PEDV genotype G2 (including the PEDV G2a and PEDV G2b genetic subtypes) could only be detected by outer primer pair I PEDV-LM-F3 and PEDV-LM-B3, and inner primer pair I PEDV-LM-FIP and PEDV-LM-BIP, but could not be detected by outer primer pair II PEDV-LS-F3 and PEDV-LS-B3, and inner primer pair II PEDV-LS-FIP and PEDV-LS-BIP. The results indicated that the optimized differential RT-LAMP method exhibited desirable specificity and repeatability for PEDV genotype G1 and PEDV genotype G2 subtype strains.

### Example 7: Sample Detection

1. One part of known PEDV genotype G1 positive sample and 1 part of known PEDV genotype G2 positive sample were each tested 10 times using the LAMP method and the PCR method, respectively.

The LAMP reaction system referred to Example 4. The PCR reaction system, in a 25 μL volume, included:
12.5 μL of a 2×PCR Master Mix,
1 μL of forward primer (SEQ ID NO: 17),
1 μL of reverse primer (SEQ ID NO: 18),
9.5 μL of sterilized distilled water, and
1 μL of sample cDNA.

The reaction program included: 94° C. for 5 min; 35 cycles of 94° C. for 10 s, 54° C. for 20 s, 72° C. for 30 s; and a final extension at 72° C. for 5 min.

The results are shown in Table 1. RT-PCR detected both the PEDV genotype G1 and genotype G2 samples in all 10 repeated tests. The universal RT-LAMP also detected both the PEDV genotype G1 and genotype G2 samples in all 10 repeated tests. However, the identification RT-LAMP could only detect the PEDV genotype G1 sample and failed to detect the PEDV genotype G2 sample.

TABLE 1
Results of RT-PCR and LAMP detection for known PEDV genotype G1 and genotype G2 strains

| | Number | Number | | | Universal | | Identification | | |
|---|---|---|---|---|---|---|---|---|---|
| PEDV | of | of | RT-PCR | | LAMP | | LAMP | | Matching |
| strain | samples | repetitions | Positive | Negative | Positive | Negative | Positive | Negative | ratio |
| PEDV G1 | 1 | 10 | 10 | 0 | 10 | 0 | 10 | 0 | 100% |
| Genotype | | | | | | | | | |
| sample | | | | | | | | | |
| PEDV G2 | 1 | 10 | 10 | 0 | 10 | 0 | 0 | 10 | |
| Genotype | | | | | | | | | |
| sample | | | | | | | | | |

2. 33 unknown suspected porcine epidemic diarrhea samples were tested for different PEDV genotypes using the LAMP method and the PCR method, respectively.

The results are shown in Table 2. Among the 33 clinical samples, LAMP detected 8 positive samples, with a positive detection rate of 24.24%. Among these, 1 sample was positive by differential LAMP, indicating that 7 samples were PEDV variant strains and 1 sample was a PEDV classical strain. In contrast, the RT-PCR method detected only 6 positive samples, with a positive detection rate of 18.18%. These results further demonstrated that the LAMP method established in the present disclosure had higher sensitivity and specificity for PEDV detection.

TABLE 2
Results of RT-PCR and LAMP detection for clinical samples

| | Number | | | Universal | | Identification | |
|---|---|---|---|---|---|---|---|
| Detection | of | RT-PCR | | LAMP | | LAMP | |
| object | samples | Positive | Negative | Positive | Negative | Positive | Negative |
| Clinical | 33 | 6 | 27 | 8 | 25 | 1 | 32 |
| sample | | | | | | | |

In summary, the LAMP detection method established in the present disclosure exhibits high sensitivity, strong specificity, excellent repeatability, and rapid detection speed. The entire process, including amplification and result determination, can be completed in just 40 min. It does not require large, complex, or expensive instruments, and result identification is convenient and visual. The kit obtained through the present disclosure not only avoids false negatives and false positives in the detection of PEDV variant strain infections but also differentiates between classical strain and variant strain of PEDV. This is of significant importance for understanding the prevalence of PEDV and for the prevention and control of PEDV infections.

Finally, it should be emphasized that the above described are only preferred embodiments of the present disclosure and are not intended to limit the present disclosure. For those skilled in the art, various changes and modifications may be made to the present disclosure, but any modifications, equivalent replacements and improvements made within the spirit and principle of the present disclosure should be included in the protection scope of the present disclosure.

1. A reverse transcription loop-mediated isothermal amplification (RT-LAMP)-based primer set for simultaneous identification of a classical strain and a variant strain of a porcine epidemic diarrhea virus (PEDV), comprising: primer set I and primer set II; wherein
primer set I comprises outer primer pair I consisting of PEDV-LM-F3 and PEDV-LM-B3, and inner primer pair I consisting of PEDV-LM-FIP and PEDV-LM-BIP; and primer set I comprises the nucleotide sequences set forth in SEQ ID NO: 1 to SEQ ID NO: 4; and
primer set II comprises outer primer pair II consisting of PEDV-LS-F3 and PEDV-LS-B3 and inner primer pair II consisting of PEDV-LS-FIP and PEDV-LS-BIP; and primer set II comprises the nucleotide sequences set forth in SEQ ID NO: 5 to SEQ ID NO: 8.

2. The RT-LAMP-based primer set according to claim 1, wherein a universal RT-LAMP-based detection primer set for detecting the classical strain and the variant strain of the PEDV is outer primer pair I and inner primer pair I from primer set I; and an RT-LAMP-based detection primer set for differentiating the classical strain and the variant strain of the PEDV is outer primer pair II and inner primer pair II from primer set II.

3. A kit for simultaneous identification of a classical strain and a variant strain of a PEDV, comprising: the RT-LAMP-based primer set according to claim 1.

4. The kit according to claim 3, further comprising a reverse transcriptase, an RT-LAMP reaction solution, a positive control, and a negative control.

5. The kit according to claim 3, wherein outer primer pair I and inner primer pair I are at a molar ratio of 1:8.

6. The kit according to claim 3, wherein outer primer pair II and inner primer pair II are at a molar ratio of 1:8.

7. A method for simultaneously differentiating a classical strain and a variant strain of a PEDV for a non-diagnostic purpose, comprising following steps:
(1) conducting RNA extraction and reverse transcription, comprising extracting a viral nucleic acid from a sample, and subjecting an RNA to reverse transcription to obtain a sample cDNA using a reverse transcription kit;
(2) conducting an RT-LAMP-based amplification reaction: preparing an RT-LAMP-based amplification reaction system by mixing the kit according to claim 3 with the sample cDNA, and conducting the RT-LAMP-based amplification reaction; and
(3) conducting result determination, comprising adding a saturated fluorescent dye to a system obtained after the RT-LAMP-based amplification reaction to conduct visual RT-LAMP-based detection, and verifying an obtained visual RT-LAMP-based detection result using agarose gel electrophoresis.

8. The method according to claim 7, wherein the RT-LAMP-based amplification reaction system comprises a universal LAMP reaction system and an identification LAMP reaction system; and
wherein the universal LAMP reaction system, in a 25 μL volume, comprises:
2.5 μL of a 10× isothermal amplification buffer,
1.0 μL of 100 mM MgSO₄,
2.5 μL of 10 mM dNTP,
1.0 μL of 8,000 U/mL Bst 2.0 DNA Polymerase,
2.0 μL of 5 μM outer primer pair I,
2.0 μL of 40 μM inner primer pair I,
2.5 μL of sample cDNA, and
11.5 μL of sterilized distilled water; and
wherein the identification LAMP reaction system, in a 25 μL volume, comprises:
1.5 μL of 100 mM MgSO₄,
3.5 μL of 10 mM dNTP,
2.0 μL of 5 μM outer primer pair II,
2.0 μL of 40 μM inner primer pair II, and
10 μL of sterilized distilled water.

9. The method according to claim 8, wherein a program for a universal RT-LAMP-based reaction comprises: a reaction at 65° C. for 40 min, a reaction at 80° C. for 20 min, and inactivation in sequence; and a program for an identification RT-LAMP-based reaction comprises: a reaction at 65° C. for 30 min, a reaction at 80° C. for 20 min, and inactivation in sequence.

10. (canceled)

11. The kit according to claim 3, wherein a universal RT-LAMP-based detection primer set for detecting the classical strain and the variant strain of the PEDV is outer primer pair I and inner primer pair I from primer set I; and an RT-LAMP-based detection primer set for differentiating the classical strain and the variant strain of the PEDV is outer primer pair II and inner primer pair II from primer set II.

12. The method according to claim 7, further comprising a reverse transcriptase, an RT-LAMP reaction solution, a positive control, and a negative control.

13. The method according to claim 7, wherein outer primer pair I and inner primer pair I are at a molar ratio of 1:8.

14. The method according to claim 7, wherein outer primer pair II and inner primer pair II are at a molar ratio of 1:8.
